(19) United States
(12) Patent Application Publication
(10) Pub. No.: US 2009/0082552 A1
(43) Pub. Date: Mar. 26, 2009

(54) Microfluidic protein assay

(76) Inventors: Magdalena Bynum, San Jose, CA (US); Timothy Beerling, San Francisco, CA (US); Brian Peter, Los Altos, CA (US); Marc Valer Serra, San Francisco, CA (US); Hui Wang, Cupertino, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/904,279

(22) Filed: Sep. 26, 2007

(51) Int. Cl. C07K 1/32; B01J 19/00; G01N 27/26

(52) U.S. Cl. 530/415; 204/600; 422/68.1

(57) ABSTRACT

A microfluidic device for separating a fluid sample into components is disclosed. The microfluidic device is used for assaying proteins for the purpose of identification of protein species. The microfluidic device employs integrated features such as a sample well for holding a protein sample, a thermal control device for heating the protein sample in the sample well, and a protein separation region in fluid communication with the sample well. Also disclosed is a method for assaying a protein sample.

### BACKGROUND

Microfluidic device technologies, also referred to as lab-on-a-chip technologies, have been proposed for a number of different applications in various fields. In the field of biology, for example, microfluidic devices may be used to carry out cellular assays. In addition, microfluidic devices have been proposed to carry out separation techniques in the field of analytical chemistry. Microfluidic technology is used in systems that perform chemical and biological analysis, as well as chemical synthesis, on a much smaller scale than previous laboratory equipment and techniques. Microfluidic systems offer the advantages of only requiring a small sample of analyte or reagent for analysis or synthesis, and dispensing a smaller amount of waste materials. A typical microfluidic channel or chamber of a microfluidic system has at least one cross-sectional dimension in the range of approximately 0.1 micrometers to 1000 micrometers. Since microfluidic technologies involve the use of small volumes of fluids, microfluidic technologies are particularly desirable in applications that involve fluids that are extremely rare and/or expensive.

One useful function served by microfluidic devices is the separation of proteins by chromatographic operations. Proteins that have been denatured, e.g. by heating, can be separated using electrophoretic chromatography; some microfluidic devices are designed to perform this separation. For example, the Bioanalyzer Protein® assay chip, sold by Agilent Technologies of Santa Clara, Calif., is used to perform microfluidic electrophoretic protein separations. A small sample of denatured protein in a buffer solution, e.g. 10 microliters or less, is loaded onto a well and a voltage is applied across a microfluidic electrophoretic separation column that is connected to the well by one or more channels, causing movement of the sample through the column. The denatured protein is then separated according to size and charge of the individual molecular species. The separated protein is then analyzed by one of a number of known techniques as it moves down the column.

However, to carry out the microfluidic separation and analysis, the protein must first be denatured and must be maintained in a denatured state prior to loading a sample onto the microfluidic device. This in turn requires the use of conventional lab benchtop techniques employing large amounts of sample compared to what is required for the microfluidic separation.

In a typical protein denaturation, the protein sample of interest is mixed with buffer and dye in an Eppendorf tube, with or without a reducing agent and a dye. A sample of interest requires a concentration of 20 nanograms per microliter to 2000 nanograms per microliter of a protein in a buffer solution, and a total sample volume of up to 100 microliters. The contents of the tube are then heated to 100° C. for about 5 minutes. Heating is typically accomplished by immersing the tube in boiling water or inserting the tube in a 100° C. heat block. After heating, the tube is spun down in a centrifuge in order to collect condensed water vapor from the walls of the tube. The protein is usually mixed with a surfactant prior to or after heating. After the heat denaturation, the sample is, in some cases, further diluted to reduce salt and detergent concentration to appropriate levels for effective separation and detection. A small sample of the denatured protein, typically 10 microliters or less, is then loaded onto the microfluidic device in order to carry out the separation and analysis of the individual molecular species.

The conventional lab scale denaturation of the protein that is required prior to microfluidic separation presents several significant disadvantages in the overall protein assay. The need to start with standard lab scale samples is a significant drawback when the protein is rare and/or expensive. The heating step can cause variations in concentration of the sample, decreasing accuracy and precision of quantification of the sample. When a heating block is used, inaccuracies in temperature control can cause deleterious effects such as protein degradation. Heater blocks are also associated with fire hazards. Additionally, the user must turn on the heat source well in advance of heating the tubes and allow the heat source to equilibrate. This step can add at least 30-45 minutes to the overall time required to get protein assay data. The handling and transfer of the sample between vessels introduces further statistical error to the overall experiment.

### BRIEF DESCRIPTION OF THE DRAWINGS

FIG. 1 is a plan view showing one embodiment of a microfluidic protein assay device.

FIG. 2 is a plan view showing another embodiment of a microfluidic protein assay device.

FIG. 3 is a plan view showing another embodiment of a microfluidic protein assay device.

FIGS. 4A-4C are cross-sectional views showing embodiments of a microfluidic protein assay device.

FIGS. 5A-5B are cross-sectional views showing additional embodiments of a microfluidic protein assay device.

FIG. 6 is a cross-sectional view showing an embodiment of microfluidic protein assay device incorporated into a system.

### DETAILED DESCRIPTION

A microfluidic device provides for the denaturation and separation of a protein sample. The microfluidic protein assay device (MPAD) includes a sample well, where a protein sample can be applied, a protein separation region connected to the sample well, and a thermal control device situated proximal to the sample well. In some embodiments, a small volume of sample, e.g. 10 microliters or less, is utilized and thus the heat source can bring the sample to the target temperature quickly, thereby denaturing the protein. Additionally, the small sample size and fast heating time provides less chance of protein degradation when compared to larger samples, which take longer to reach the target temperature and thus are subjected to a longer total heating time.

In some embodiments, the thermal control device is embedded within the MPAD itself. In other embodiments the heat source is located on a separate apparatus. The thermal control device can be in contact with the sample when the sample is disposed within the sample well, or it can be embedded in the MPAD such that there is a spacer between the sample well and the thermal control device. When mounted on a separate apparatus, the MPAD is exposed to the thermal control device by contacting the thermal control device situated on a separate substrate. This may be accomplished by providing, for example, a recess in the side of the MPAD substrate opposite to the side where the sample well is defined, such that the thermal control device fits inside the recess and is disposed in close proximity to the sample well when the MPAD is mounted on the apparatus.

In some embodiments, the thermal control device is capable of either adding or removing heat from a sample. Thermoelectric devices are known in the art. Providing an electric current in one direction through the thermoelectric device results in the addition of heat to a sample. Reversing current through the thermoelectric device results in the removal of heat. Such thermal control devices are useful where rapid and/or controlled cooling of the sample after heating is desirable.

Upon loading a protein sample into the sample well of the MPAD, the thermal control device heats the protein sample. Upon reaching a target temperature, the protein sample becomes denatured. The denatured protein is then directed to the protein separation region. Typically, the sample well is connected by a channel to the protein separation region. Typically, the protein separation region is a microfluidic electrophoretic separation column. The protein separation region can be loaded with a standard electrophoretic separation medium. A voltage applied across the electrophoretic separation column causes the sample to move from the sample well to the protein separation region and through the protein separation region, separating the denatured protein into individual protein species. The separated protein species are analyzed, using an external detector commonly used to detect protein species.

Combining protein denaturation with microfluidic separation on a microfluidic device presents several surprising and unexpected advantages in the overall protein assay when compared to traditional protein denaturation followed by protein separation. The ability to start with very small scale protein samples is significant particularly when the protein is rare and/or expensive. The heating of a small sample is faster and uses less energy than heating conventional lab scale samples. Additionally, the reduced overall time during which a protein sample is exposed to heat results in less protein degradation, which in turn leads to an increase in accuracy and precision in quantification of the sample. The handling and transfer of the sample between vessels is obviated, eliminating a source of contamination and further reducing statistical error in the overall experiment.

Additional advantages and novel features of the invention will be set forth in part in the description that follows, and in part will become apparent upon examination of the following, or may be learned through routine experimentation upon practice of the invention.

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

In one embodiment, a microfluidic protein assay device (MPAD) has a substrate in which are defined features that include a sample well, a thermal control device proximate to the sample well, and a protein separation region in fluid communication with the sample well. The features collectively occupy an area of the substrate of about 0.1 to 10 cm². The sample well receives a protein sample and the thermal control device heats the sample. In some embodiments, a protein sample is retained within the sample well while the thermal control device heats the sample. In some embodiments, upon reaching a target temperature, the sample is urged into the protein separation region where the denatured protein is separated into various molecular species, which are then available for analysis. In other embodiments, the sample is cooled prior to being urged into the protein separation region.

Substrate

In some embodiments, the substrate in which microfluidic features are defined is a substrate known in the art to be suitable for the fabrication of microfluidic devices. Examples of suitable substrates include glass, silicon, and plastic. In some embodiments, polydimethylsiloxane (PDMS) is the substrate. Some advantages of PDMS are that it is inexpensive, optically clear, and permeable to several substances, including gases. Since air can quickly diffuse out, the latter aspect is very convenient, making it possible to inject fluid into a channel that has no outlet. In other embodiments, poly(ether ether ketone) (PEEK) is the substrate. Advantages associated with PEEK include excellent mechanical properties and resistance to thermal degradation. Glass and polyimide are other commonly used substrate materials in microfluidic applications.

Protein separation region

Various chromatographic techniques of separating molecular species using a microfluidic device are disclosed in U.S. Pat. Nos. 7,128,876, 6,702,256, and 6,958,119, which are incorporated herein by reference. In some embodiments, the protein separation region is a column having a length of about 0.5 cm to about 5 cm. In some embodiments, the protein separation region is an electrophoretic protein separation column, where the electrophoretic protein separation column includes a polyacrylamide gel, an agarose gel, a cellulose acetate polymer, or another solid medium suitable for protein separation. The medium can be a crosslinked polymer. Any known medium used to carry out electrophoretic separation is useful in such embodiments; the electrophoretic separation column is not limited by the type of medium used to facilitate separation of denatured proteins. It will be understood that the separation medium is selected depending on the type and/or molecular weight of the protein to be separated.

Suitable solid phase media for electrophoretic separations of proteins includes sieving matrices, such as, but not limited to polyacrylamide, agarose, dextran or combinations thereof. In such embodiments, the solid phase media is generally prepared as a gel matrix and includes an electrically-conductive buffer solution. A variety of these and other suitable solid media for electrophoresis of proteins are commercially available.

In some embodiments, the solid phase for electrophoretic separation is a polyacrylamide gel. In these embodiments, the polyacrylamide medium includes polyacrylamide at between about 0.5% and 25% by weight, between about 7% and 20% by weight, between about 10% and 15% by weight, or about 12% by weight. In embodiments the polyacrylamide is prepared as a gel that includes an electrically-conductive buffer solution. In an embodiment, the polyacrylamide gel is non-denaturing to proteins. In another embodiment, the polyacrylamide gel is protein denaturing. Denaturing poyacrylamide gels include denaturants, such as detergents, salts, or combinations thereof. In embodiments, a denaturing polyacrylamide gel includes a concentration of sodium dodecyl sulfate (SDS) typically between about 0.01% and 5% by weight, between about 0.1% and 3% by weight, between about 0.2% and 1% by weight, or about 0.25% by weight.

In other embodiments, the solid medium for electrophoretic separation is purified agarose. In some such embodiments, the solid phase includes purified agarose at concentration between about 0.1% and 2.5% by weight, between about 0.5% and 1.5% by weight, or between about 0.8% and 1.1% by weight. In embodiments the agarose is prepared as a gel and includes an electrically conductive buffer solution.

In embodiments where the protein separation region is an electrophoretic separation column, the MPAD has two or more electrodes situated to apply an electrical voltage across the separation column. The electrical voltage, when applied, serves to urge a denatured protein sample through the column, thereby separating the denatured protein into individual molecular species.

In some embodiments, the protein separation region has a first end and a second end, wherein the first end is in fluid connection with the sample well, and the second end is where the sample ends up after elution through the protein separation region. In some embodiments, the MPAD has an additional well in fluid communication with the second end of the protein separation region to collect fluids issuing from the protein separation region.

In embodiments, the protein separation region of the MPAD includes a selected length, width, and depth. In embodiments, the protein separation region is about 75 mm to 1 mm long, about 50 mm to 5 mm long, or about 25 mm to 10 mm long. In embodiments, the protein separation region is about 2 μm to 25 μm deep, about 7 μm to 18 μm deep, or about 11 μm to 14 μm deep. In embodiments, the protein separation region is about 5 μm to 100 μm wide, about 20 μm to 80 μm wide, or about 30 μm to 50 μm wide. In embodiments, the dimensions of the protein separation region are selected according to the type and/or molecular weight of the protein to be separated. A longer protein separation region typically allows for finer resolution of the proteins in the sample, while a shorter protein separation region typically allows for quicker sample throughput.

Reagents

In some embodiments, the MPAD can have a source of reagents. In some embodiments the reagents are preloaded into the MPAD prior to addition of a protein sample. Such reagents are, in embodiments, reagents suitable for protein denaturation, protein separation, protein detection, or a combination of these. Thus, the reagents include, in some embodiments, one or more buffer solutions, detergents, dyes or fluorescent molecules and/or standard proteins.

In some embodiments, one or more of the reagents are present in one or more additional wells defined on the MPAD substrate. In some embodiments, one or more additional wells are in fluid connection with the sample well. In other embodiments one or more additional wells are in fluid communication with the protein separation region. In yet other embodiments one or more additional wells are in fluid communication with both the sample well and the protein separation region.

In some embodiments, a detergent or a detergent solution is present in an additional well that is in fluid connection with the sample well, so that the detergent can be added to a protein sample. In some embodiments the detergent is added to the protein sample after denaturation and prior to the sample reaching the protein separation region. In other embodiments, the detergent is added to the protein sample prior to denaturation. In embodiments, the detergent is present in a well on the MPAD that provides detergent solution into the sample prior to or after heating the sample.

Typically, a detergent is provided in a solution of water or buffer. The choice of detergent is not particularly limiting to the various embodiments disclosed herein. In some embodiments, sodium dodecyl sulfate is the detergent. However, in other embodiments, other anionic surfactants, nonionic surfactants such as, for example, Triton® X-100 ((C₁₄H₂₂O(C₂H₄O)ₙ)) or Tween®-20 (polyoxyethylene sorbitan monolaurate) (available from the Sigma-Aldrich Company of St Louis, Mo.), or zwitterionic detergents such as, for example, 3-[(3-cholamidopropyl) dimethylammonio]-1-propanesulfonate (CHAPS), are used as the detergent. A detergent is used in some embodiments to associate with the denatured protein so as to prevent association of the denatured protein strands. In some embodiments, detergents are added to the protein samples after denaturation and prior to separation.

In other embodiments, a set of standard proteins is present in an additional well that is in fluid connection to the protein separation region, such that the standard proteins are provided to the protein separation region for elution through the protein separation region. The standard proteins, in some embodiments, are present in a buffer solution. Standard proteins that are employed in some embodiments include one or more of bovine serum albumin, albumin, cytochrome C, myoglobin, and carbonic anhydrase. However, in other embodiments different standard proteins or combinations thereof are used. The choice of standard proteins is not particularly limiting to the various embodiments disclosed herein. In embodiments, standard proteins are employed in the MPAD as a co-elution standard of known molecular weight, against which an unknown protein sample can be measured for an accurate comparative determination of molecular weight. Standard proteins may be pre-labeled with fluorescent dyes or other detection molecules or they may be detected in the same manner as the proteins in the sample.

In some embodiments, chromophores and fluorescent dyes are used as internal standard for determination of molecular weight. For example, in embodiments, chromophores or small molecules are selected based on their molecular weight and mass-to-charge ratio. These markers are validated against various protein standards of known molecular weight and mobility in the separation assay. Small molecular weight standards can have the advantages of stability over protein standards, and thus may increase the consistency and ease of use of the assay.

In some embodiments, a detection agent such as a dye, fluorescent molecule or other detection label is used to aid in detection of the sample and/or standard proteins. Non-limiting examples of dyes include Coomassie® Brilliant Blue or SYPRO® Ruby, available from Invitrogen Corporation of Carlsbad, Calif., USA or Krypton™ protein stain available from Pierce Biotechnology, Rockford, Ill., USA. Non-limiting examples of fluorescent labeling molecules include NHS-Fluorescein, NHS-Rhodamine, or the Dylight™ protein labeling dyes available from Pierce Biotechnology, Rockford, Ill., USA. Examples of other detection labels include quantum dots available from Invitrogen Corporation, Carlsbad, Calif., USA. The detection agent may be covalently or noncovalently associated with the proteins in the sample or the standards. Suitable protein detection agents and methodologies are employed in conjunction with these embodiments.

In some embodiments, the MPAD employs one or more buffers. The choice of buffer employed with the MPAD is not particularly limited and should be chosen based on the protein being assayed. Examples of buffers commonly employed in embodiments are tris-HCl, urea, glycine, or mixtures of these at various values of pH (Sigma-Aldrich Company of St Louis, Mo.). In some embodiments, a buffer is used to maintain a constant concentration of the protein; in other embodiments it is used to elute the protein sample within the protein separation region; in still other embodiments it is used for both purposes. In embodiments, a buffer is used to transport one or more detergents, one or more standard proteins, or one or more other reagents within the MPAD. In other embodiments, the buffer can be present in an additional well that is in fluid connection with both the sample well and the protein separation region, such that the buffer is added to the denatured protein sample, for example, during denaturation, and buffer is also added to the denatured sample as it is eluted through the protein separation region.

Microfluidic Channels

In some embodiments, the substrate of the MPAD defines fluid connections between features that are microfluidic channels. For example, in embodiments, a microfluidic channel connects the sample well and the protein separation region. In other embodiments, a microfluidic channel connects the sample well and an additional well holding a standard protein sample. In yet other embodiments, a microfluidic channel connects the protein separation region to an additional well holding a reagent. In still other embodiments, a microfluidic channel connects the protein separation region and an additional well disposed to hold materials that have eluted through the protein separation region.

Typically, in embodiments, microfluidic channels connecting features on the device have a width of about 10 micrometers to about 100 micrometers and a depth of about 5 micrometers to about 50 micrometers.

Formation of Microfluidic Features

Microfluidic channels, protein separation regions, sample wells, additional wells, and other microfluidic features are not limited by the technique with which they are formed. Microfluidic channels can be made by any known technique including lithography, laser etching, printing, and microreplication of inverse images of features disposed on PDMS, nickel, or polyimide onto a final substrate such as a thermoplastic. In some embodiments, microreplication is carried out by a melt technique, such as applying heat and pressure to a thermoplastic disposed against a mold having an inverse pattern. In other embodiments, microreplication can be carried out by casting an uncured polymer such as PDMS onto a mold and curing the polymer, followed by removal of the mold. In other embodiments, glass is patterned by photolithography before channels are wet or dry etched. Any of these techniques, or others, can be used to form microfluidic channels on the substrate of the MPAD. The described techniques are merely illustrative and do not limit the techniques that can be used to make microfluidic channels.

Thermal control device

The thermal control device is situated proximal to the sample well and heats a protein sample placed in the sample well to denature the protein prior to separation. In some embodiments, the thermal control device is embedded within the MPAD. In other embodiments the thermal control device is external to the MPAD but situated nearby so that it can heat a protein sample in the sample well.

In some embodiments, the thermal control device is embedded within the MPAD itself. In some such embodiments, the thermal control device is situated so as to be in direct contact with a protein sample within the sample well. In other embodiments, the thermal control device is situated within the MPAD but not such that has direct contact with the protein sample within the sample well. In some embodiments where the thermal control device is proximate to, but not in direct contact with, the protein sample within the sample well, the thermal control device is situated about 25 μm to 500 μm from a bottom portion of the sample well. In other embodiments, the thermal control device is situated about 100 μm from a bottom portion of the sample well. In some embodiments, the thermal control device is situated in the side of the substrate opposite the side in which the features are defined. Such a configuration thus defines the spacing between the thermal control device and the sample well, and also provides convenient access to the thermal control device such that a source of electricity can be easily connected to it.

In some embodiments the thermal control device embedded in the MPAD is in the form of a ring that surrounds the sample well. In other embodiments the embedded thermal control device is in the form of a cylinder having one closed end, such that the thermal control device encloses the sample well. In other embodiments the embedded thermal control device is circular, oval, or rectilinear in shape and is situated immediately below the sample well. In some embodiments, an embedded thermal control device matches one or more dimensions of the sample well. In other embodiments the embedded thermal control device is smaller than one or more dimensions of the sample well. In still other embodiments the embedded thermal control device is larger than one or more dimensions of the sample well.

In some embodiments, the thermal control device is situated external to the MPAD. In some embodiments, such external thermal control device is located adjacent the side of the MPAD substrate in which the sample well is defined. In other embodiments, the external thermal control device is located adjacent the side of the MPAD substrate in which the sample well is defined. In still other embodiments, the external thermal control device is located beside the sample well.

In some embodiments, a recess is defined in the MPAD substrate in the side of the MPAD substrate opposite the side in which the sample well is defined and proximate to the sample well. The recess is shaped to receive a thermal control device. In these embodiments, when the MPAD is situated within a machine having a thermal control device, the thermal control device is external to the MPAD. The external thermal control device is situated within the recess proximate to the sample well. In some embodiments, the external thermal control device is a thermoelectric device. In other embodiments, the external thermal control device is a resistive device joule heater). In some embodiments, the thermal control device is a microheater chip. In other embodiments, the external thermal control device is an electromagnetic heating device, for example, an infrared emitting device or a microwave emitting device, that is disposed above or below the side of the MPAD substrate in which the sample well is defined. Where the electromagnetic heating device is a microwave emitting device, water present in the protein sample converts microwave radiation emitted by the microwave emitting device to heat. Where the electromagnetic heating device is an infrared emitting device, the MPAD additionally includes a structure, such as a black body, that absorbs the infrared radiation emitted by the infrared emitting device and converts the absorbed radiation into heat. One example of a suitable infrared emitting device is an infrared laser. Additionally or alternatively, the electromagnetic heating device may emit electromagnetic energy in other regions of the electromagnetic spectrum, e.g., visible light.

In some embodiments, the thermal control device is capable of heating a sample placed in the sample well to a target temperature of up to about 90° C. or 105° C. In other of these embodiments, the thermal control device is capable of heating a sample placed in the sample well to the target temperature at a rate of about 1° C./sec to 10° C./sec. In still other of these embodiments, the thermal control device is capable of maintaining the target temperature of a sample within the sample well to within about 0.1° C. to 2° C.

In embodiments where the thermal control device is a thermoelectric device, a source of electric current supplies the thermoelectric device, such that, depending on a direction of current flow through the thermoelectric device, the thermoelectric device can provide either addition of heat to or removal of heat from a sample in the sample well. The ability of a thermoelectric device to both add heat and actively remove heat (as opposed to passive cooling obtained simply by no longer heating a sample at an above-ambient temperature) provides additional control over the temperature profile used to denature a protein sample in the MPAD. For example, in some embodiments, the thermoelectric device can remove heat from the sample at a rate that provides a temperature change of about 1° C. to about 10° C./sec.

Valves

In some embodiments, the MPAD includes one or more valves. The valves are, in embodiments, mechanical valves such as those described in U.S. Pat. No. 6,702,256, which is incorporated herein by reference in its entirety. In other embodiments, the MPAD has valves including a thermoelectric device capable of providing either addition of heat or removal of heat, and a material capable of phase change upon addition of heat or removal of heat by the thermoelectric device. Valves employing thermoelectric devices and phase changing materials are described in U.S. Pat. Nos. 6,007,302 and 5,975,856, which are incorporated herein by reference in their entirety.

In embodiments, the material capable of phase change is a sample disposed between the sample well and the protein separation region, for example in a channel connecting the sample well and the protein separation region. In other embodiments, the material capable of phase change is an additional material provided within the MPAD.

In embodiments, the valve may assist in urging a sample more quickly or more slowly into and through a protein separation region by e.g. heating the sample, or cooling to freeze the sample when the sample is disposed within a channel; in other embodiments a mechanical valve assists in urging a sample more quickly or more slowly into and through a protein separation region.

### Other Embodiments

Other embodiments encompassing variations of the elements described above are also useful. For example, in some embodiments, an MPAD having multiple sample wells and multiple protein separation regions is used to analyze multiple proteins in a single experiment. By forming fluid connections between sets of sample wells and protein separation regions such that each set is discrete, cross contamination of samples is avoided. Similarly, in other embodiments, multiple protein separation regions are used to simultaneously elute standard proteins and a denatured protein samples, again avoiding cross contamination. Other embodiments are envisioned without departing from the scope of the invention.

In some embodiments, the microfluidic device is part of a system. The system includes, in embodiments, a module for holding the microfluidic device. In such embodiments, the module includes a thermal control device; thus the module is a thermal module in such embodiments. In embodiments, the system includes one or more clamps to hold the microfluidic device to the module. In embodiments, the system includes an optical interrogation device for analyzing separated protein samples. In embodiments, the optical interrogation device is in data communication with a computer that provides data interpretation of information provided by the optical interrogation device. In other embodiments, the thermal control device is in data connection with a computer that provides regulation of a thermal cycle. In still other embodiments, both the optical interrogation device and the thermal control device is in data connection with a computer or with respective computers.

Methods of Separating Proteins

In embodiments, in a method of denaturing and separating proteins, a microfluidic protein assay device (MPAD) is provided. The MPAD includes a substrate defining features. The features include at least one sample well having a thermal control device proximate to the sample well, and a protein separation region in fluid communication with the well. The features collectively occupy an area of the substrate less than or equal to about 0.1 cm² to 10 cm². A protein sample is applied to the MPAD. The protein sample is heated by the thermal control device to about 90° C. to 105° C. for at least 5 minutes in the presence of a detergent to form a detergent-protein complex. The detergent-protein complex is urged toward the protein separation region. The detergent-protein complex is separated.

In some embodiments, the method further involves addition of at least one source of reagents to the MPAD. The reagents include, in embodiments, a buffer, a detergent, and an assay standard protein. Providing reagents in this manner obviates the need to admix reagents into the sample before applying the sample to the sample well of the MPAD. In some embodiments, the applying involves applying about 1 picoliter to 10 microliters of the protein sample to the MPAD. When reagents necessary to carry out the denaturation and separation are preloaded onto the MPAD, the applying of the sample requires no further additives to be mixed into the sample.

In some embodiments, the method further involves analyzing the separated detergent-protein complex. In some embodiments, the analyzing is accomplished optically, for example, by ultraviolet measurement, infrared measurement, or fluorescence measurement. Fluorescence measurements are carried out by, for example, loading a fluorescent tag bearing chemical as reagent within a well on the MPAD, such that the tag can become associated with the protein. Such associations allow quantification of fluorescence on a protein molecule that in turn allows quantification of molecular weight of individual species by measuring the level of fluorescent emissions of a species as it is eluted on the separation column.

### DETAILED DESCRIPTION OF THE FIGURES

Turning to the Figures, certain exemplary embodiments will be described.

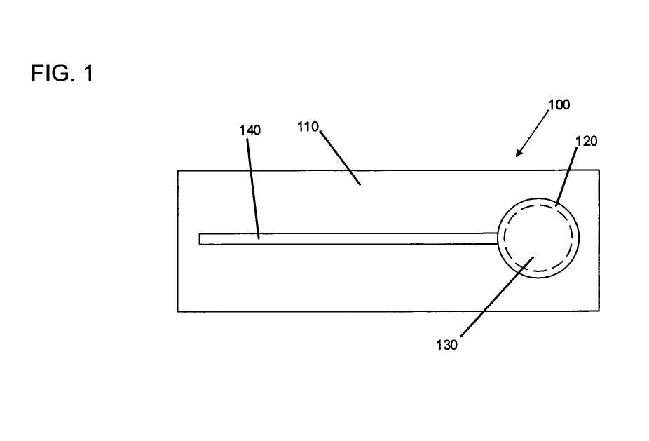

FIG. 1 is a plan view showing an example of an MPAD 100. The MPAD 100 includes a substrate 110, a sample well 120, a thermal control device 130 disposed beneath sample well 120, and a protein separation region 140.

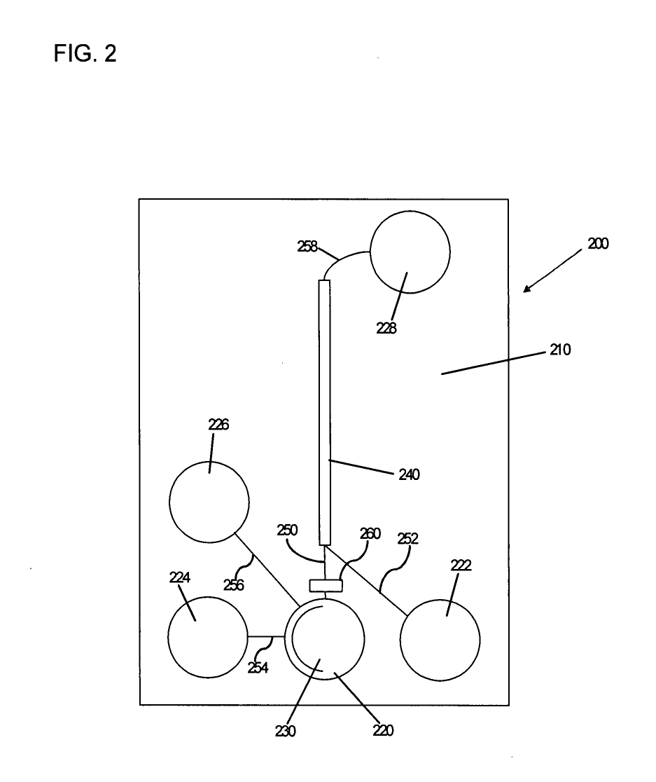

FIG. 2 is a plan view showing an example of an MPAD 200. The MPAD 200 includes a substrate 210, a sample well 220, a thermal control device 230 disposed beneath sample well 220, and a protein separation region 240. Also included are additional wells 222, 224, 226, 228 and channels 250, 252, 254, 256, 258. Additional well 222 is connected by channel 252 to protein separation region 240. Additional well 220 is connected by channel 250 to protein separation region 240. Additional well 224 is connected by channel 254 to sample well 220. Additional well 226 is connected by channel 256 to sample well 220. Channel 250 further has a valve 260 disposed in communication therewith.

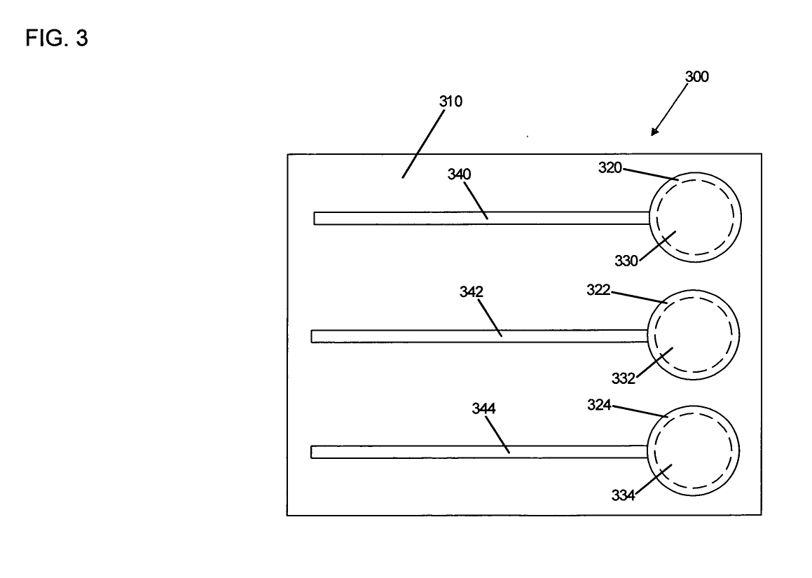

FIG. 3 is a plan view showing an example of an MPAD 300. MPAD 300 includes a substrate 310, sample wells 320, 322, 324, a thermal control device 330 disposed beneath sample well 320, a thermal control device 332 disposed beneath sample well 322, and a thermal control device 334 disposed beneath sample well 324. MPAD 300 additionally includes a protein separation region 340 in fluid communication with sample well 320, a protein separation region 342 in fluid communication with sample well 322, and a protein separation region 344 in fluid communication with sample well 324.

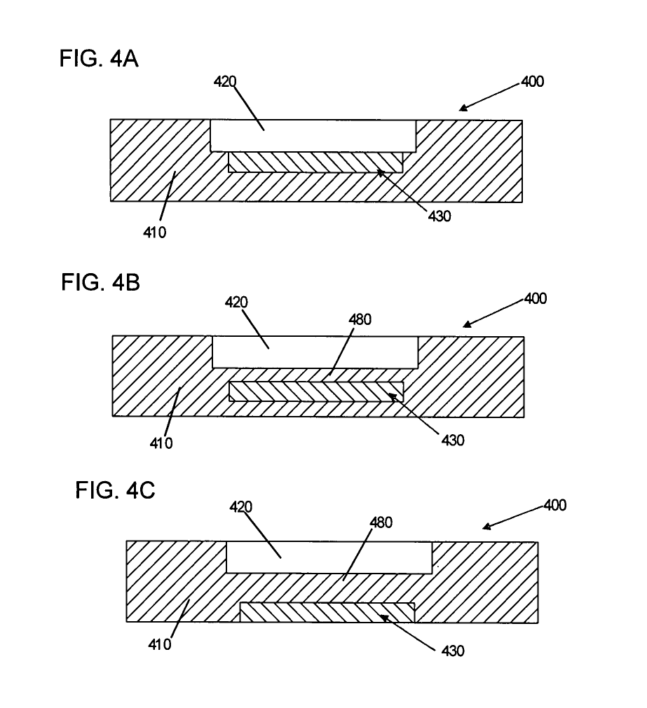

FIGS. 4A-4C are cross-sectional views showing part of an example of an MPAD 400. Visible in FIG. 4A is a substrate 410, a sample well 420, and a thermal control device 430 disposed beneath sample well 420. Thermal control device 430 is disposed so as to be in direct contact with a sample within sample well 420.

FIG. 4B shows a similar embodiment to FIG. 4A, except that thermal control device 430 is disposed proximate to, but not in direct contact with, a bottom portion of sample well 420. In this embodiment, a spacer 480 is located between thermal control device 430 and the bottom portion of sample well 420; thus, a sample within sample well 420 does not directly contact thermal control device 430 because of spacer 480.

FIG. 4C shows a similar embodiment to FIG. 4B, except that thermal control device 430 is located on the side of substrate 410opposite the side of substrate 410 in which sample well 420 is defined. Thus, spacer 480 in this embodiment is thicker than the spacer 480 shown in FIG. 4B. Further, thermal control device 430 is exposed to the external surface of MPAD 400.

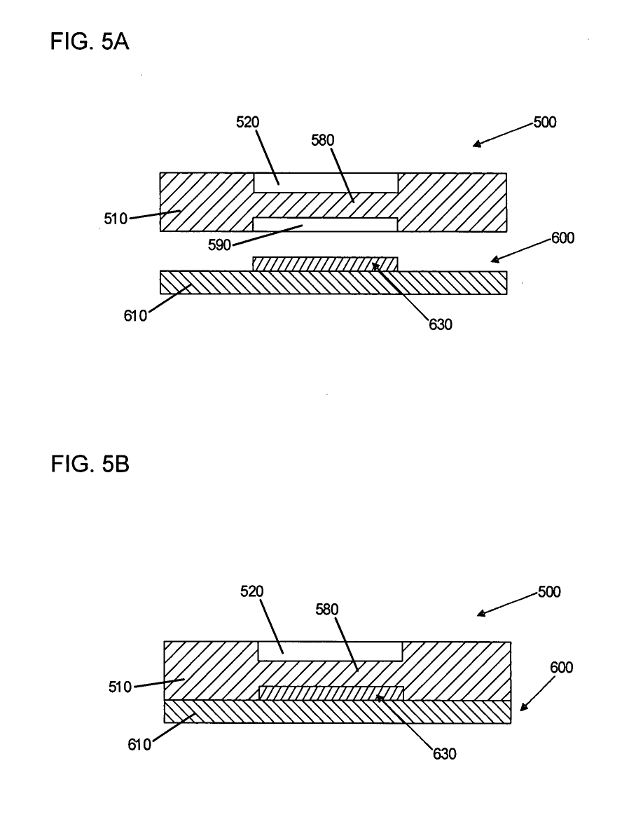

FIGS. 5A and 5B are cross-sectional views showing part of another example of an MPAD 500. Visible in FIG. 5A is a substrate 510 and a sample well 520. A spacer 580 is located between sample well 520 and a recess 590. Recess 590 is a hollowed out region of substrate 510. Also visible is thermal control device 630 disposed externally to MPAD 500 and located on a substrate 610 of a thermal control module 600. The dimensions of recess 590 correspond to the dimensions of thermal control device 630. Thus, in FIG. 5B, it can be seen that, when MPAD 500 and thermal module 600 are contacted, thermal control device 630 is disposed within recess 590 such that thermal control device 630 is in direct contact with the substrate 510 of MPAD 500. Thermal control device 630 is further disposed beneath well 520 and separated from the sample well 520 by spacer 580.

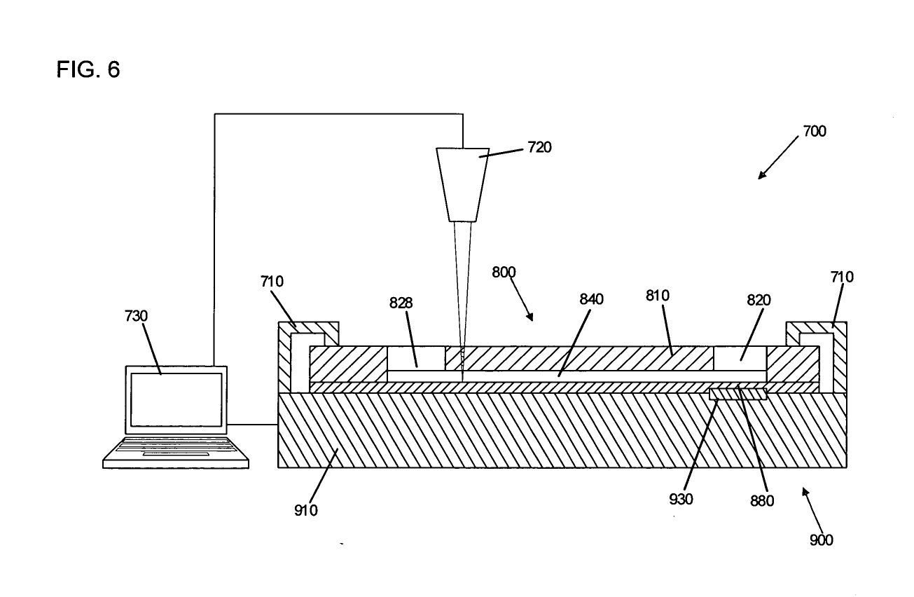

FIG. 6 is a cross-sectional view showing an example of an embodiment of an apparatus 700 including an MPAD 800 and a thermal control module 900. MPAD 800 includes a substrate 810, a sample well 820, a protein separation region 840 in fluid communication with sample well 820, an additional well 828 in fluid communication with protein separation region 840, and a spacer 880. Thermal module 900 includes a substrate 910 and a thermal control device 930. Thermal control device 930 is in direct contact with substrate 810 of MPAD 800 such that thermal control device 930 is disposed beneath sample well 820 and is separated from the bottom portion of sample well 820 by spacer 880. In embodiments, thermal control device 230 is a thermoelectric device, and thus is capable of both heating and cooling a sample in sample well 820.

Apparatus 700 further includes clamps 710 affixed to thermal module 900 and serving to hold MPAD 800 within apparatus 700. An optical interrogation module 720 is disposed above protein separation region 840 such that separated components of denatured protein samples traveling from protein separation region 840 to well 828 are analyzed by optical interrogation carried out by optical interrogation module 720 as they travel. In embodiments, optical interrogation module 720 facilitates an optical measurement that includes, for example, a ultraviolet measurement, an infrared measurement, or a fluorescence measurement. In embodiments, optical interrogation module 820 is in data connection with an external computer 730, which, in embodiments, interprets signals from optical interrogation module 720. In embodiments, external computer 730 is also in data communication with thermal module 900 and provides signals to control heating of sample well 820 by thermal control device 930.

The various embodiments described above are provided by way of illustration only and should not be construed to limit the claims attached hereto. Various modifications and changes may be made without following the exemplary embodiments and applications illustrated and described herein, and without departing from the scope of the following claims.

The present invention may suitably comprise, consist of, or consist essentially of, any of the disclosed or recited elements. Thus, the invention illustratively disclosed herein can be suitably practiced in the absence of any element which is not specifically disclosed herein.

What is claimed:

1. A microfluidic protein assay device, comprising:
a substrate defining a sample well and a protein separation region in fluid communication with the well, the sample well and the protein separation region collectively occupying an area of the substrate of about 0.1 to 10 cm²; and
a thermal control device positioned proximal to the sample well.

2. The microfluidic protein assay device of claim 1 wherein the substrate comprises glass, silicon, or poly(ether ether ketone).

3. The microfluidic protein assay device of claim 1 wherein the substrate additionally defines one or more additional wells in fluid communication with one or both of the sample well and the protein separation region.

4. The microfluidic protein assay device of claim 3, wherein:
one or more reagents are disposed in one or more of the additional wells, the reagents comprising one or more of a buffer, a detergent, and a set of standard proteins; and
the detergent comprises sodium dodecyl sulfate or lithium dodecyl sulfate and the set of standard proteins comprise one or more of bovine serum albumin, albumin, cytochrome C, myoglobin, and carbonic anhydrase.

5. The microfluidic protein assay device of claim 1, wherein the protein separation region comprises an electrophoretic separation column.

6. The microfluidic protein assay device of claim 1, wherein:
the protein separation region has a first end and a second end;
the first end is in fluid communication with the sample well;
the substrate further defines a second well;and
the second well is in fluid communication with the second end of the protein separation region.

7. The microfluidic protein assay device of claim 1, wherein the substrate additionally defines a channel connecting the well to the protein separation region.

8. The microfluidic protein assay device of claim 7, further comprising a valve in communication with the channel.

9. The microfluidic protein assay device of claim 1, wherein the thermal control device is capable of heating a sample in the well to a target temperature of up to about 90° C. to 105° C.

10. The microfluidic protein assay device of claim 9, wherein the thermal control device is capable of heating a sample in the sample well to the target temperature at a rate of about 1° C./sec to 10° C./sec.

11. The microfluidic protein assay device of claim 9, wherein the thermal control device is capable of maintaining the sample at the target temperature to within about 0.1° C. to 2° C.

12. The microfluidic protein assay device of claim 9, wherein the sample is retained within the sample well while the thermal control device heats the sample.

13. The microfluidic protein assay device of claim 1, wherein the thermal control device comprises one of a thermoelectric device, a resistive heating device, and an electromagnetic heating device.

14. The microfluidic protein assay device of claim 13, wherein the thermal control device comprises a thermoelectric device that can add heat to or remove heat from a sample in the sample well depending on a direction of current flow through the thermoelectric device.

15. The microfluidic protein assay device of claim 14, wherein the thermoelectric device can remove heat from the sample at a rate that reduces the temperature of the sample at a rate of about 1° C./sec to about 10° C./sec.

16. The microfluidic protein assay device of claim 1, wherein the thermal control device is embedded within the substrate.

17. The microfluidic protein assay device of claim 1, wherein the substrate additionally defines a spacer about 25 μm to 500 μm thick interposed between the thermal control device and a bottom portion of the sample well.

18. The microfluidic protein assay device of claim 17, wherein spacer is about 100 μm thick.

19. The microfluidic protein assay device of claim 1, wherein:
the sample well is a first sample well;
the protein separation region is a first protein separation region;
the substrate additionally defines a second sample well and a second protein separation region; and
the second sample well is in fluid communication with the second protein separation region independently of the first sample well and the first protein separation region.

20. A method of separating proteins, the method comprising:
providing a microfluidic protein assay device comprising:
a substrate defining a sample well and a protein separation region in fluid communication with the sample well, the sample well and the protein separation region collectively occupying an area of the substrate of about 0.1 to 10 cm², and
a thermal control device positioned proximal to the sample well;
applying a protein sample to the sample well;
heating the protein sample on the sample well to about 90° C. to 105° C. for at least 5 minutes in the presence of a detergent to form a detergent-protein complex sample;
urging the detergent-protein complex sample toward the protein separation region; and
separating the detergent-protein complex sample.

21. The method of claim 20, further comprising adding one or more reagents to the protein sample, the reagents comprising one or more buffers, detergents, detection agents, and assay standard proteins.

22. The method of claim 20, wherein the applying comprises applying about 1 picoliter to 10 microliter of the protein sample to the sample well.

23. The method of claim 20, further comprising analyzing the separated detergent-protein complex.

24. The method of claim 23, wherein the analyzing is accomplished optically.

25. The method of claim 24, wherein the analyzing comprises an ultraviolet measurement, an infrared measurement, or a fluorescence measurement.
